(19) United States
(12) Patent Application Publication
(10) Pub. No.: US 2013/0089869 A1
(43) Pub. Date: Apr. 11, 2013

(54) Methods For and Uses of Mechanical Stiffness Profiling of Cancer Cells

(71) Applicant: Duke University, Durham, NC (US)

(72) Inventors: Gerard C. Blobe, Durham, NC (US); Mythreye Karthikeyan, Durham, NC (US)

(73) Assignee: DUKE UNIVERSITY, Durham, NC (US)

(21) Appl. No.: 13/646,057

(22) Filed: Oct. 5, 2012

Related U.S. Application Data
(60) Provisional application No. 61/543,633, filed on Oct. 5, 2011.
(60) Provisional application No. 61/576,730, filed on Dec. 16, 2011.

(51) Int. Cl. G01N 33/50

(52) U.S. Cl. CPC G01N 33/5044; G01N 33/5005
USPC 435/7.1; 435/29

(57) ABSTRACT

Methods of predicting the invasiveness or metastatic potential of cancer cells are provided herein. Methods of screening for cancer cells or diagnosing cancer in a subject are also provided. Methods of screening for agents capable of reducing invasiveness or metastasis of cancer cells are also provided. All of the methods rely on analyzing the creep compliance or spring constant of cells.

### CROSS-REFERENCE TO RELATED APPLICATIONS

This patent application claims the benefit of priority of U.S. Provisional Patent Application No. 61/543,633, filed Oct. 5, 2011 and U.S. Provisional Patent Application No. 61/576,730, filed Dec. 16, 2011, which are both incorporated herein by reference in their entirety.

### STATEMENT REGARDING FEDERALLY SPONSORED RESEARCH

This invention was made with United States government support awarded by the National Institutes of Health grant number R01 CA1135006. The United States may have certain rights in this invention.

### INTRODUCTION

This invention relates to the fields of cancer diagnostics, cancer prognostics, and assays for screening cancer therapeutics.

The spread of cancer from its primary site to distant organs, the “invasion-metastasis cascade”, is the main cause of cancer death and invasion of cells into the lymphatics and blood vessels is a crucial step in metastasis, correlating with a poorer patient prognosis. Hallmarks of invasion include secretion of proteases, alterations in adhesion receptors, and changes in cell morphological and migratory properties. Drugs targeting the metastatic cascade, including the matrix metalloproteinases (MMPs), which degrade the extracellular matrix, or the migratory machinery, are being evaluated in clinical trials but results have been disappointing potentially due to the complexity and redundancy of the metastatic cascade.

### SUMMARY

In the Examples, cellular mechanical stiffness is related to the invasiveness of cancer cells and their metastatic potential. Methods of predicting the invasiveness of a cancer cell are provided herein. The methods include determining the cell stiffness by measuring the creep compliance (deformability) or the spring constant (stiffness) of the cancer cell and using this determination to predict the invasiveness of the cancer cell. The creep compliance of the cell is proportional to the invasiveness of the cell and the spring constant is inversely proportional to the invasiveness of the cell.

In another aspect, methods of predicting the metastatic potential of a cancer cell are provided. The methods include determining the creep compliance (deformability) or the spring constant (stiffness) of the cancer cell and using this determination to predict the metastatic potential of the cancer cell. The creep compliance of the cell is proportional to the metastatic potential of the cell and the spring constant is inversely proportional to the metastatic potential of the cell.

In yet another aspect, methods of screening for an agent capable of reducing the invasiveness of a cancer cell are provided. The methods include contacting the cancer cell with the agent and determining the creep compliance or spring constant of the cell after contact with the agent. An agent capable of decreasing the creep compliance or increasing the spring constant of the cell as compared to a second cancer cell not contacted with the agent is an agent capable of reducing the invasiveness of the cancer cell.

In a still further aspect, methods of screening for cancer cells are provided The methods include measuring the creep compliance (deformability) or the spring constant (stiffness) of a cell from a subject to predict whether the cell is cancerous. In this method, high creep compliance of the cell is predictive of the cell being cancerous and low spring constant is predictive of the cell being cancerous.

In yet a further aspect, methods of diagnosing cancer in a subject are provided. The methods include measuring the creep compliance (deformability) or the spring constant (stiffness) of a cell from a subject to predict whether the cell is cancerous. A high creep compliance of the cell is predictive of the cell being cancerous and a low spring constant is predictive of the cell being cancerous.

### BRIEF DESCRIPTION OF THE DRAWINGS

FIG. 1 is a set of pictorial depictions showing several typical methods for force application in cell biology.

FIG. 2 is a set of graphs showing invasive cancer cells have the highest compliance. A) Invasion assays were performed on the indicated cancer cell lines (Methods) and percent invasion (I) relative to each other presented. Data represent the mean±SEM of three independent experiments. B) invasion of primary cancer cells as described in (A). C) Maximum compliance for all the cell lines is presented. The three boxes encompassed by dashed lines indicate the different scored regions based on relative invasion, with cell lines within a box not being statistically significant from each other mechanically (p≧0.05). (Dash dot box (--)—high invasion, I≧0.4, Solid box (-)—medium invasion, 0.2<I<0.4, dashed box (---) low invasion, I≦0.2). D) Maximum compliance for primary cancer cells is presented. The boxes separate statistically significant groups of primary cancer cells similar to (C). All mechanical measurements represent mean±SEM.

FIG. 3 is a set of graphs showing that stiffness correlates with invasion. Stiffness was calculated by fitting a modified Kelvin Voigt model (FIG. 6) to compliance curves for ovarian cancer cell lines (A) and primary ovarian cancer cells (B) and mapped with the relative invasion from FIGS. 2A and 2B. The boxes represent the same scored regions as in FIGS. 2C and 2D. C.) Power law showing the correlation between the stiffness of ovarian cancer cell lines and their invasion. IGROV when treated with blebbistatin (open circle, solid arrow) and Ovca429Neo with Ovca429TβRIII (triangle, dash dot arrow) move on the line, consistent with the correlation. (INSET) Power law on log log plot. D) Power law correlation for the primary ovarian cancer cells. (INSET) power law on a log log plot.

FIG. 4 is a set of photographs and graphs showing that highly invasive and stiff cancer cells express cortical actin and myosin. Immunoflourescence images of cells stained either for (A) actin using rhodamine conjugated to phalloidin or with an antibody to (B) phosphorylated myosin light chain (pMLC). Quantification of fluorescence intensity using Image J software across the lines shown in the corresponding panels on the left are indicative of stress fiber density in the case of actin or cortical pMLC localization. C) Stiffness of the respective ovarian cancer cell lines.

FIG. 5 is a set of graphs showing Myosin II function and TβRIII alter stiffness and invasion. A) Invasion assays of Ovca429-Neo and Ovca429-TβRIII were performed as described in the Methods and FIG. 2. Data are a composite of two independent experiments performed in duplicate. Each column represents the mean±SEM. B) Stiffness for the corresponding cell type in (A) obtained as described in the Methods and FIG. 3. C) Effect of blebbistatin treatment on the invasion of IGROV and Ovca429-TβRIII cell types. D) Stiffness for the corresponding cell type and treatments in (C) (**−p<0.01, *=p<0.05).

FIG. 6 shows a micrograph of the magnetic beads in conjunction with the cells (A), a graph showing the resultant radial displacement of the beads after force application (B), a graph showing the fitting of a modified Kelvin Voight model to the compliance (C) and the derivation of spring constants by fitting the compliance curves to a Jeffrey's model for viscoelastic liquids (D).

FIG. 7 shows a graph of the percentage of cell migration of the various cell lines (A) and a second graph showing the relationship between cell migration and cell stiffness (B).

FIG. 8 is a set of micrographs and graphs showing staining of actin (A) and pMLC (B) in IGROV cells and supporting a relationship between actomyosin contractility, cell stiffness and invasion.

FIG. 9 is a graph showing the distribution of all cell stiffness values for a less invasive cell (IGROV) and a highly invasive cell (Skov3). The distribution of cell stiffness was very distinct.

FIG. 10 is a Western blot showing the expression levels of E-cadherin, vimentin and β-actin in the various cells.

### DETAILED DESCRIPTION

A variety of biophysical techniques including membrane stretching, atomic force microscopy, optical traps and micropipette aspiration have been used to probe the mechanical properties of cells. These techniques use ferromagnetic or super paramagnetic beads to attach to membrane receptors and are followed by application of either a twisting or a pulling motion to the bead and thus to the cell via an electromagnet. Magnetic tweezers, like the one described here, provide for a wide range of force magnitudes (10 pN-10 nN) to be measured, the ability to probe individual cells and to perform measurements in minutes to understand the time dependent development of a cell's mechanical state. While cancer tissue has been found to be generally stiffer than normal tissue, recent studies have shown that cancer cells themselves are more compliant than normal cells. However, the extent of the correlation between mechanical properties and specific aspects of cancer progression has not been determined. In the Examples, cellular mechanical stiffness is demonstrated to be related to the invasiveness of cancer cells and their metastatic potential.

Methods of predicting the invasiveness or metastatic potential of a cancer cell and methods of screening for agents capable of reducing the invasiveness or metastatic potential or metastasis of cancer cells are provided herein. The methods include determining the cell stiffness by measuring the creep compliance, also referred to herein as deformability, or the spring constant, also referred to herein as stiffness, of a cancer cell. The measurements can then be used to predict the invasiveness or metastatic potential of the cancer cell and the cancer from which the cell was obtained. In the Examples, the creep compliance and spring constant of cancer cells is demonstrated to be predictive of the invasiveness of the cell or cancer being tested. The creep compliance of the cell or cancer is proportional to the invasiveness of the cell, such that higher creep compliance indicates the cell is more invasive and has a higher likelihood of being or becoming metastatic. The spring constant of the cell is inversely proportional to the invasiveness or metastatic potential of the cell or cancer, such that a lower spring constant indicates the cell or cancer is more invasive and has a higher likelihood of being or becoming metastatic.

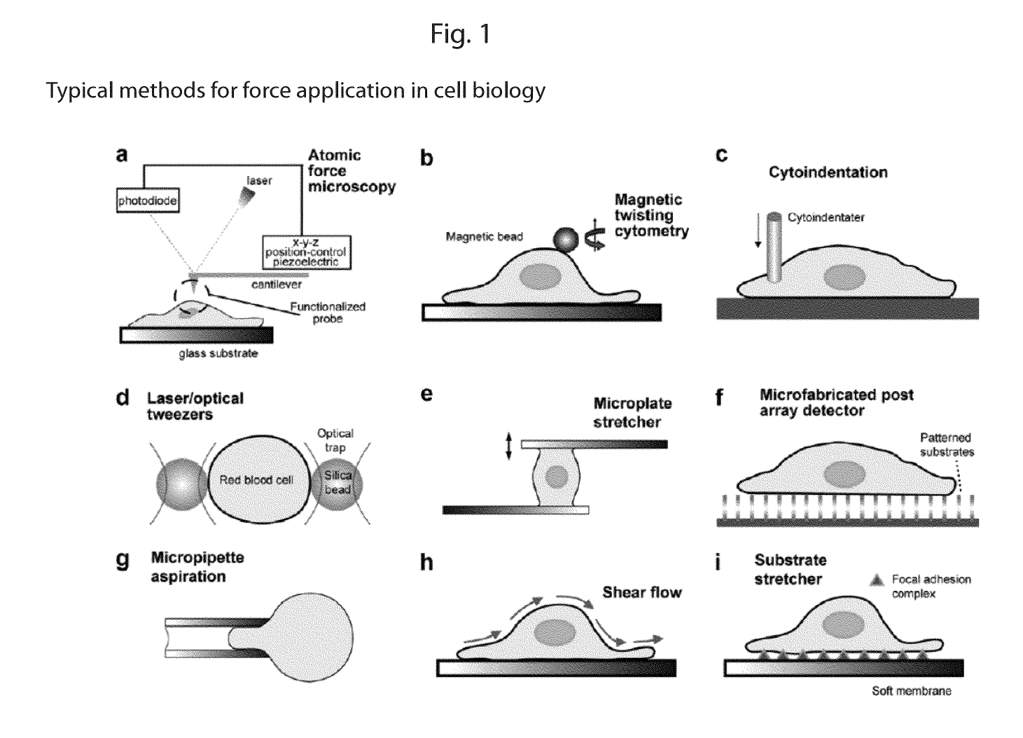

The creep compliance and spring constant of cells may be measured using a variety of techniques. In the Examples, magnetic tweezers were used. As shown in FIG. 1, several other methods may be used as well, including but not limited to, atomic force microscopy, micropipette aspiration, microfluidic optical stretcher, laser or optical tweezers, shear flow, substrate stretcher or a microplate stretcher. These methods are described at least in the following references: Lekka M, Laidler P. Nat. Nanotechnol. 2009 February; 4(2):72; Substrate flexibility regulates growth and apoptosis of normal but not transformed cells. Wang H B, Dembo M, Wang Y L. Am J Physiol Cell Physiol. 2000 November; 279(5):C1345-50. Optical deformability as an inherent cell marker for testing malignant transformation and metastatic competence. Guck J. Schinkinger S, Lincoln B, Wottawah F, Ebert S, Romeyke M, Lenz D, Erickson H M, Ananthakrishnan R, Mitchell D, Ka's J, Ulvick S, Bilby C. Biophys J. 2005 May; 88(5):3689-98. Epub 2005 Feb. 18. Sun D, Wang J, Yao W, Cu L, Wen Z, Shu C. Clin Hemorheol Microcirc. 2004; 30(2):117-26. Tumorigenesis of murine erythroleukemia cell line transfected with exogenous p53 gene. Each of the references cited herein is incorporated herein by reference in its entirety.

The creep compliance or spring constant of the cell is then used to form or generate a prediction regarding the invasiveness or metastatic potential of the cell and the cancer from which the cell was obtained. “Predicting” and “prediction” as used herein includes, but is not limited to, generating a statistically based indication of whether a particular cell or cancer is likely to be invasive or metastatic. This does not mean that the event will happen with 100% certainty. The prediction is meant to allow a gradation of a cancer as being more or less aggressive, i.e. likely to become invasive and exhibit metastasis. The predictions are based on the creep compliance and/or spring constant numbers obtained for the cell in which higher creep compliance numbers and lower spring constants are associated with higher invasiveness and an increased metastatic potential, as described in more detail below. The predictions may also be based on comparison of the creep compliance and/or spring constant of a cell with those of comparable cells from cancers with known invasiveness or metastatic potential.

Cellular mechanical stiffness was measured as shown in the Examples. Briefly, the creep compliance (deformability) was calculated as the average time dependent deformation normalized by the constant stress applied

$$( {J}_{max} = \frac{{r}_{max} \times 6 π a}{F} ,$$

where a is the radius of the bead and rₘₐₓ is maximum bead displacement). In the Examples, a creep compliance (Jₘₐₓ) greater than 1 is shown to correlate or be indicative of increased invasiveness or increased metastatic potential. Thus a creep compliance (Jₘₐₓ) of more than 1, 1.5, 2, 2.5, 3 or more is predictive of the cell or the cancer from which the cell was associated or derived is highly invasive, has increased potential for developing metastases and is an aggressive cancer. In contrast, a creep compliance (Jₘₐₓ) of less than 0.5, 0.4, 0.3, 0.2, 0.1 or less is predictive of the cell or the cancer from which the cell was associated or derived is not highly invasive, has decreased potential for developing metastases and is not an aggressive cancer.

The stiffness may also be measured by measuring the spring constant. The effective shear modulus (herein referred to as the stiffness or spring constant, k), of the cell was calculated by fitting a modified Kelvin Voigt model to the compliance using a least squares fit (See Bausch et al., Biophys J 1999; 76: 573-579). A spring constant (k) of less than 2 was correlated with and indicative of increased invasiveness or increased metastatic potential. Thus a spring constant (k) of less than 2, 1.5, 1, 0.75, 0.5 or less is predictive of the cell or the cancer from which the cell was associated or derived is highly invasive, has increased potential for developing metastases and is an aggressive cancer. In contrast, a spring constant (k) of more than 2.5, 3.0, 3.5, 4.0, 5, 6, or more is predictive of the cell or the cancer from which the cell was associated or derived is not highly invasive, has decreased potential for developing metastases and is not an aggressive cancer.

The method and prediction of metastatic potential or invasiveness of a cancer cell may be used to provide a prognosis to a subject from whom the cancer cell was obtained. The prediction regarding the invasiveness or metastatic potential and the prognosis may be used to develop a treatment regimen, wherein a more aggressive treatment regimen for the cancer is developed for subjects with a more invasive cancer or a cancer with a high metastatic potential. Alternatively, a less aggressive therapeutic treatment regimen could be recommended for subjects whose cancer cells are found to be less invasive and have a lower metastatic potential. The treatment plans provided herein may result in treatment of the cancer.

Treating cancer includes, but is not limited to, reducing the number of cancer cells or the size of a tumor in the subject, reducing progression of a cancer, maintaining a cancer in a less aggressive form, reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with a disease or at risk of developing the disease, including improvement in the condition of the subject (e.g., in one or more symptoms), delay in the progression of the disease, delay the onset of symptoms or slow the progression of symptoms, etc.

As used herein, “individual” and “subject” are interchangeable. A “patient” refers to an “individual” who is under the care of a treating physician. Application of the treatment regimen may result in treatment of the subject with the cancer. Subjects include mammals, suitably humans. Suitably, the subjects are subjects diagnosed with cancer or suspected of having cancer.

In the methods described herein, the cancer cell may be obtained from biopsy, ascites, tumor, urine, sputum, pleural fluid or circulating cells. The cells may be obtained from a subject using any method known in the art, including from a needle aspiration or tumor resection procedure. The cells may be obtained from any solid tumor and may contain non-cancerous cells. Suitably, at least 40%, 50%, 60%, 709%, 80%, 90%, 95%, 98%, or 99% of the cells in the sample are cancer cells. In preferred embodiments, samples having greater than 50% cancer cell content are used. In one embodiment, the sample is a live tumor or cellular sample obtained from the subject. In another embodiment, the sample is a frozen sample. In one embodiment, the sample is one that was frozen within less than 5, 4, 3, 2, 1, 0.75, 0.5, 0.25, 0.1, or 0.05 hours after extraction from the patient. Frozen samples include those stored in liquid nitrogen or at a temperature of about −80° C. or below. The cells may be from a cancer including but not limited to ovarian, breast, uterine, lung, colon, pancreatic, prostate, stomach, thyroid, skin, melanoma, liver, esophagus, head and neck, bladder, sarcoma, cervical or kidney cancer cells. The cells may be obtained from a solid tumor or tissue using means available to procure cells available to those of skill in the art.

Methods of screening for cancer cells comprising measuring the creep compliance (deformability) or the spring constant (stiffness) of a cell from a subject to predict whether the cell is cancerous are also provided. Cells with high creep compliance are likely to be cancerous and those cells with a low spring constant are likely cancerous. Methods of diagnosing cancer in a subject are also provided. As above, the creep compliance (deformability) or the spring constant (stiffness) of a cell from a subject may be used to predict whether the cell is cancerous. The subject being screened for cancer or diagnosed in these methods may have been treated for cancer prior to the cells being used in the method. In other embodiments, the subject is at risk of developing cancer or is suspected of having cancer and the method is used to detect or diagnose cancer. Cancer cells may be identified by comparison to control non-cancerous cells or control cancer cells.

Methods of screening for an agent capable of reducing the invasiveness of a cancer cell are also provided. The methods involve contacting the cancer cell with the agent and determining the creep compliance or spring constant of the cell after contact with the agent. An agent capable of decreasing the creep compliance or increasing the spring constant of the cell as compared to a second cancer cell or control cell not contacted with the agent is an agent capable of reducing the invasiveness or metastatic potential of the cancer cell. The second cell or control cell is suitably a matched cell from the same line or cancer as the cell being contacted with the agent.

The cells for use in screening assays may be cancer cells such as those described above and may include primary cells or cell lines known to be invasive or have a high metastatic potential. Cells may be contacted with the agent directly or indirectly in vivo, in vitro, or ex vivo. Contacting encompasses administration to a cell, tissue, mammal, patient, or human. Further, contacting a cell includes adding an agent to a cell culture. Other suitable methods may include introducing or administering an agent to a cell, tissue, mammal, or patient using appropriate procedures and routes of administration as defined above. Some agents may require administration in or with a delivery vehicle. Suitable delivery vehicles are available to those of skill in the art.

The methods of measuring the stiffness of the cells after treatment with the agent are described above and include measuring the creep compliance and/or the spring constant. The methods used are as described above. An agent capable of decreasing the creep compliance by two fold, three-fold or four or more fold is indicative of an agent capable of reducing the invasiveness of the cancer cell. An agent capable of increasing the spring constant by two-fold, three-fold, four-fold or more is indicative of an agent capable of reducing the invasiveness of the cancer cell. Such agents may be useful as treatments for cancer or as candidates for treatment of cancer by reducing metastatic potential or invasiveness.

The following examples are meant only to be illustrative and are not meant as limitations on the scope of the invention or of the appended claims. All references cited herein are hereby incorporated by reference in their entireties.

### EXAMPLES

### Materials and Methods.

### Cell Culture and Reagents:

Human ovarian cancer cell lines, OVCA429, IGROV, SKOV3, HEY, DOV13, OV2008, Ovca420 and ovarian cancer stable cells lines, Ovca429Neo, Ovca429 TβRIII were cultured, derived, and characterized as previously described⁹. Antibody to pMLC (myosin light chain) was obtained from Cell Signaling Technologies (Cat. No. 3671) and pan-cytokeratin antibody was obtained from Santa Cruz (Cat. No. 81714).

Isolation of Cancer Cells from Ascites:

Primary short term epithelial ovarian cancer cell cultures were established from the ascites of patients with Stage III/IV epithelial ovarian cancer as described previously¹⁰. Cells were seeded and grown on 10 μg/mL fibronectin coated culture dishes in RPMI media containing 20% FBS and 1% penicillin/streptomycin solution at 37° C. in 5% CO₂. Adhered cells were subject to limited dispase digestion for the first passage to remove fibroblasts and stained with a pan-cytokeratin antibody to confirm epithelial origin.

### Immunoflourescence:

Immunoflourescence was performed essentially as described previously⁹ and images were obtained using a Nikon inverted microscope.

### Matrigel Invasion and Transwell Migration Assays:

Cancer cells were seeded at a cell density of 25,000-70,000 on either Matrigel coated or uncoated filters and allowed to invade for 18-24 hours towards 10% FBS in the lower chamber. Cells invading and migrating through the Matrigel layer were visualized and counted as described⁹. Percent cell migration or invasion was determined as the fraction of total cells that invaded through the filter. Blebbistatin (100 μM)¹⁰ where used, was added to the top chamber of the transwell and migration and invasion allowed to proceed. Each assay was set up in duplicate, and each experiment was conducted at least 3 times with 4 random fields from a 10× magnification analyzed for each membrane.

### Magnetic Tweezers Assay:

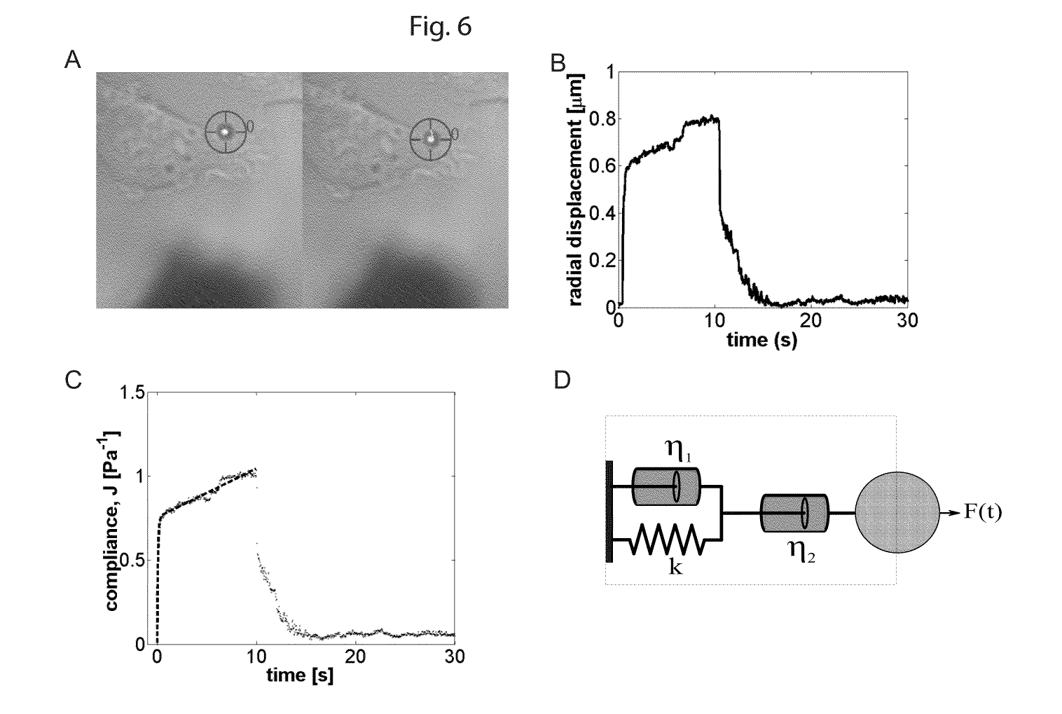

The Three Dimensional Force Microscope (3DFM)¹¹ was used for applying controlled and precise 60-100 pN local force (FIG. 6) on 2.8 micron magnetic beads (DYNAL Biotech, Oslo, Norway) coated with fibronectin (Sigma Aldrich, St. Louis, Mo.). Briefly, cells were plated on coverslips followed by addition of the beads. Cells and beads were incubated for 30 minutes followed by force application and resultant bead displacements were recorded and analyzed. The displacement of the beads was recorded with high-speed video camera (Pulnix, JAI, Ca) and tracked using Video Spot Tracker (http://cismm.cs.unc.edu). The mean creep compliance was calculated from the tracked displacements as described. Spring constants were derived by fitting the compliance curves to a Jeffrey's model for viscoelastic liquids (FIG. 6). For pharmacological experiments, blebbistatin, (100 μm) was added to the cells for 30 mins, prior to addition of the beads and the reagent left in for the remainder of the experiment.

### Results and Discussion:

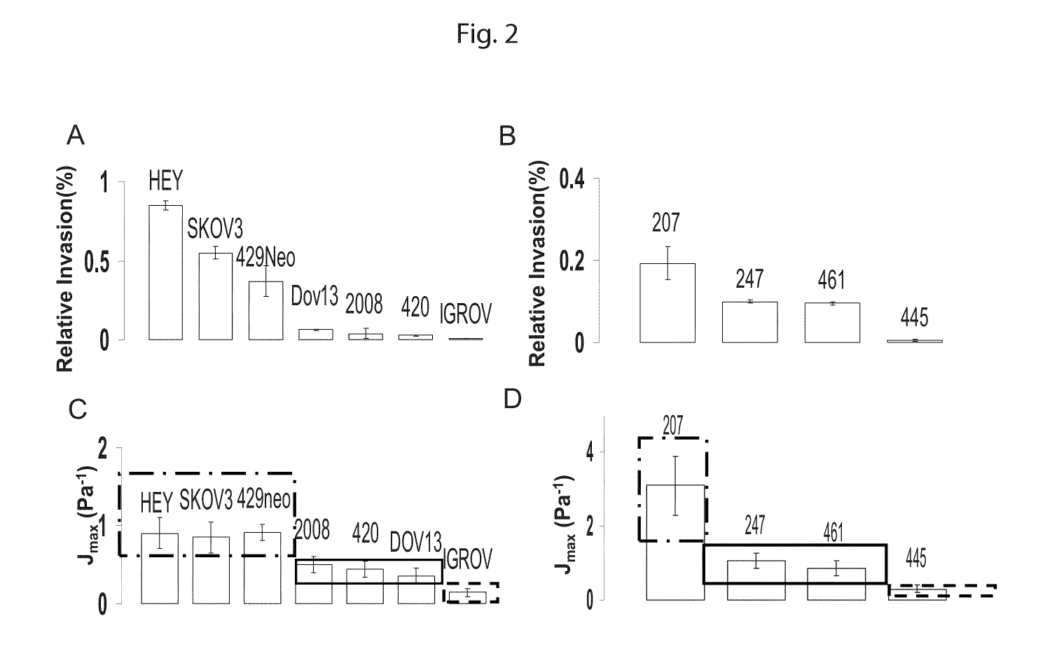

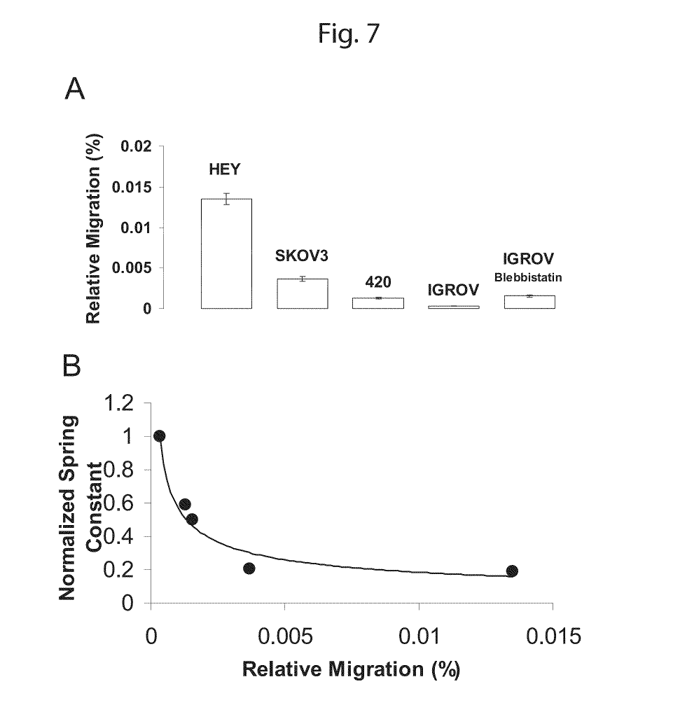

The invasiveness and migratory capacity of a panel of ovarian cancer cell lines and primary cells derived from ascites of patients with advanced stage ovarian cancer was determined using transwell assays in the presence or absence of reconstituted Matrigel (Methods, FIG. 7). While both ovarian cancer cell lines and primary cancer cells were able to invade through Matrigel, the degree of invasiveness varied widely among individual lines, with the most invasive and migratory cell line, HEY, being two orders of magnitude more invasive (I_{HEY}=0.85%, I_{IGROV}=0.006%) than the least migratory and invasive cell line, IGROV (FIG. 2A). Similarly, while primary cells were obtained from patients at either stage III or stage IV disease (Table 1), the most invasive primary cell line, OV207, was an order of magnitude more invasive than the least invasive primary cell line, OV445 (FIG. 2B; I_{OV207}=0.193%, I_{OV445}=0.006%).

TABLE 1
Description of Primary Cells

| Primary cell ID | Stage | Grade | Histology |
|---|---|---|---|
| OV247 | IIIC | 3 | Serous |
| OV461 | III | 2 | Serous |
| OV445 | III | 2 | Serous |
| OV207 | IV | 2 | Serous |

Mechanical properties of the cancer cells from the same passage as used for invasion studies were determined in parallel using a three dimensional force microscope (3DFM) based magnetic tweezer system¹¹. The creep compliance (deformability) was calculated as the average time dependent deformation normalized by the constant stress applied

$$( {J}_{max} = \frac{{r}_{max} \times 6 π a}{F} ,$$

where a is the radius of the bead and rₘₐₓ is maximum bead displacement). We find that the most invasive cell line, HEY, was 10 times more deformable than the least invasive cell line IGROV. In addition, OV207 that exhibited 30 fold greater invasion than OV445, had a Jₘₐₓ=3.1 Pa⁻¹ in contrast with the Jₘₐₓ=0.3 Pa⁻¹ observed for OV445 (FIGS. 2C and 2D). Hence both cell lines and primary cells that exhibited high invasive behavior also presented high Jₘₐₓ values and were more compliant.

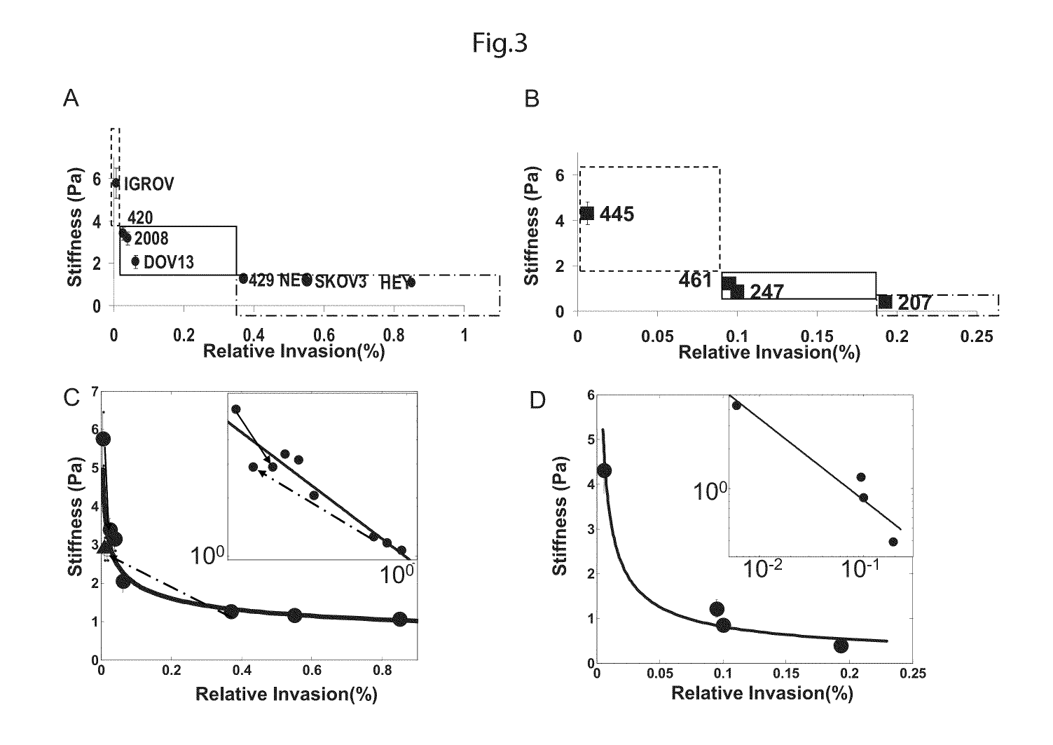

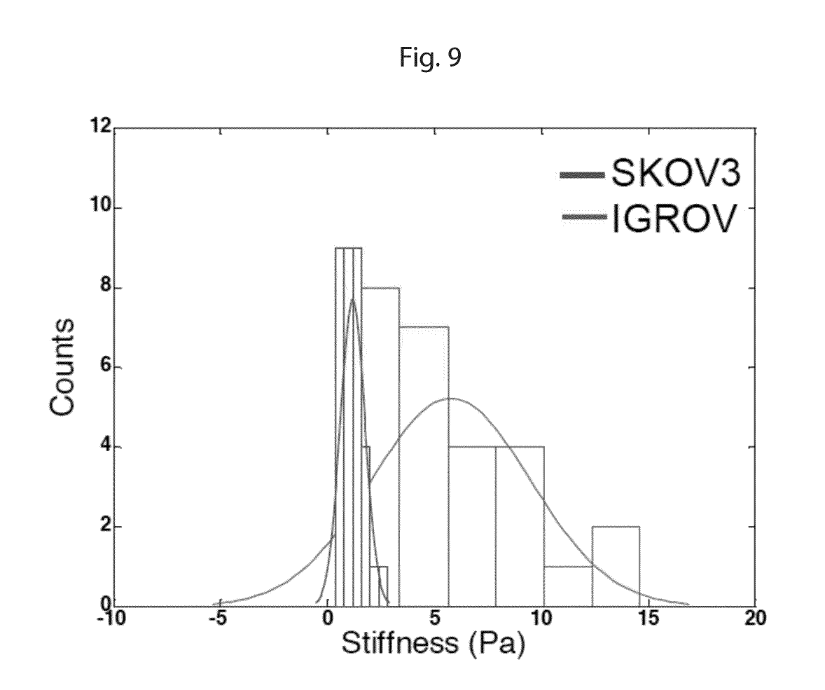

To further examine the relationship between cancer cell deformability and invasive potential, the effective shear modulus (here on referred to as the stiffness, k), of the cell was calculated by fitting a modified Kelvin Voigt model¹² to the compliance using a least squares fit (see FIG. 6). The cancer cell lines and primary cells were classified both by their stiffness and their invasiveness, with both parameters falling into three classes of low, medium and high stiffness or invasiveness, respectively. Cell lines within a given class did not exhibit statistically significant differences in their stiffness, while cell lines between classes, were significantly different (p≦0.05). Consistent with previous findings, the distribution of all stiffness values for the less invasive cell line (IGROV) showed a log normal distribution whereas the highly invasive cell line (Skov3) showed a normal distribution (FIG. 9)⁸. Scaling the cell line and primary cell correlations separately with their respective highest cell stiffness values resulted in a single parameter power law (FIG. 3D). A similar correlation and power law was also observed for stiffness and cell migration (FIG. 7) (p_{cell lines}=−0.95 and pₚᵣᵢₘₐᵣᵢₑₛ=0.96 in log log scale). While previous reports have demonstrated alterations in cell stiffness of cancer cells either from body fluids or tumors¹³, our data using ovarian cancer cell lines and cells from patient ascites demonstrate that cancer cells across a given disease population exhibit a varying degree of stiffness, a phenomenon previously not described. In addition, the variability in stiffness correlates directly with a specific measure of metastatic progression as determined using in vitro three-dimensional invasion assays.

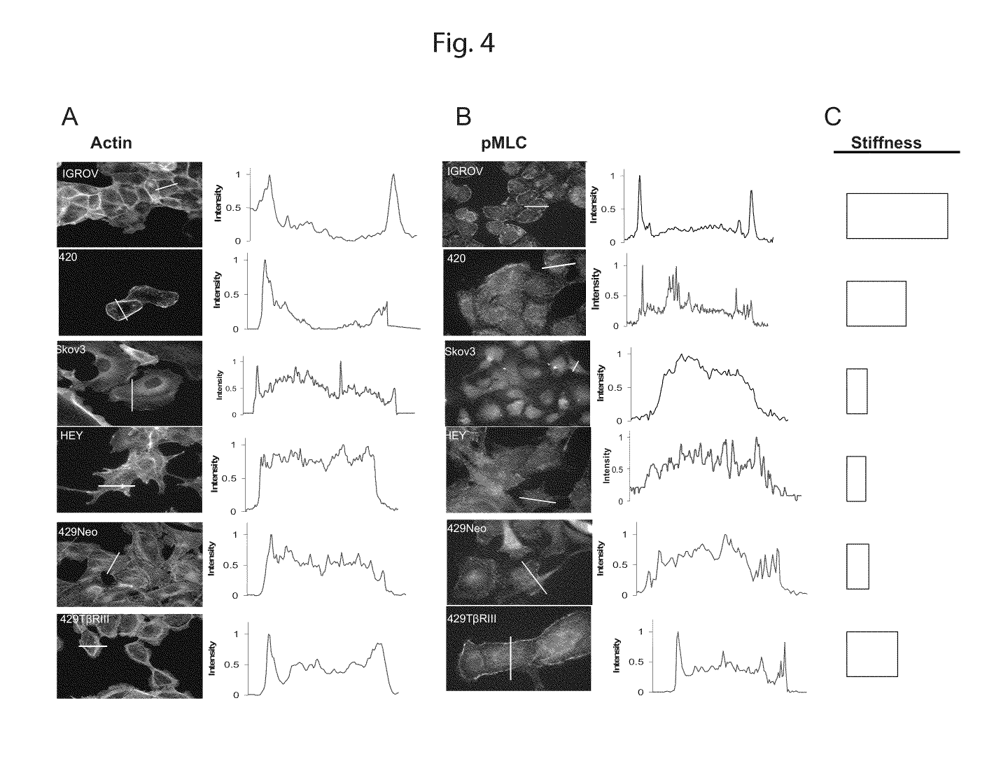

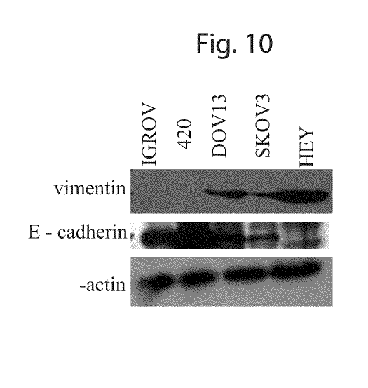

Stiffness and deformation are strongly regulated by actomyosin contractility^{14,15}. Phosphorylation of the 20 kD regulatory myosin light chain (MLC) subunit on the Ser19 (mono) or on Ser19/Thr18(di)¹⁶ has been shown to promote cell contractility via changes in the actin myosin network¹⁷. Visualization of actin in the stillest and least invasive cell line. IGROV, revealed strong cortical n staining with little to no cell protrusions or lamellipodial structures (FIG. 4A). In contrast, the compliant and invasive cell lines, including SKOV and HEY cells, exhibited distinct lamellipodial and protrusive structures with limited cortical actin. In addition, phosphorylated myosin light chain (pMLC) was found distinctly along the cell periphery in IGROV's (FIG. 4B) while SKOV3 and HEY cells had little to no peripheral pMLC localization. This phenotypic difference between the stiffest/least invasive and the compliant/most invasive cell lines might reflect differences in epithelial character of the cells. Accordingly, we examined the expression of an epithelial marker E-Cadherin) and a mesenchymal marker (Vimentin) in these cell lines. Indeed, the stiffest/least invasive cell lines expressed more E-Cadherin and less Vimentin, while the compliant/most invasive cell lines expressed less E-Cadherin and more Vimentin (FIG. 10). Intriguingly, while the Ovca420 and IGROV cells both exhibited high cortical actin (FIGS. 4A and 4B), high E-Cadherin expression and low Vimentin expression (FIG. 10), they exhibited a 1.7 fold difference in stiffness, which corresponded to a 4-fold difference in invasion (FIGS. 2 and 3). These data demonstrate that cell stiffness measurements performed as described may be a more discerning measurement of metastatic potential than examining cell structure or epithelial character.

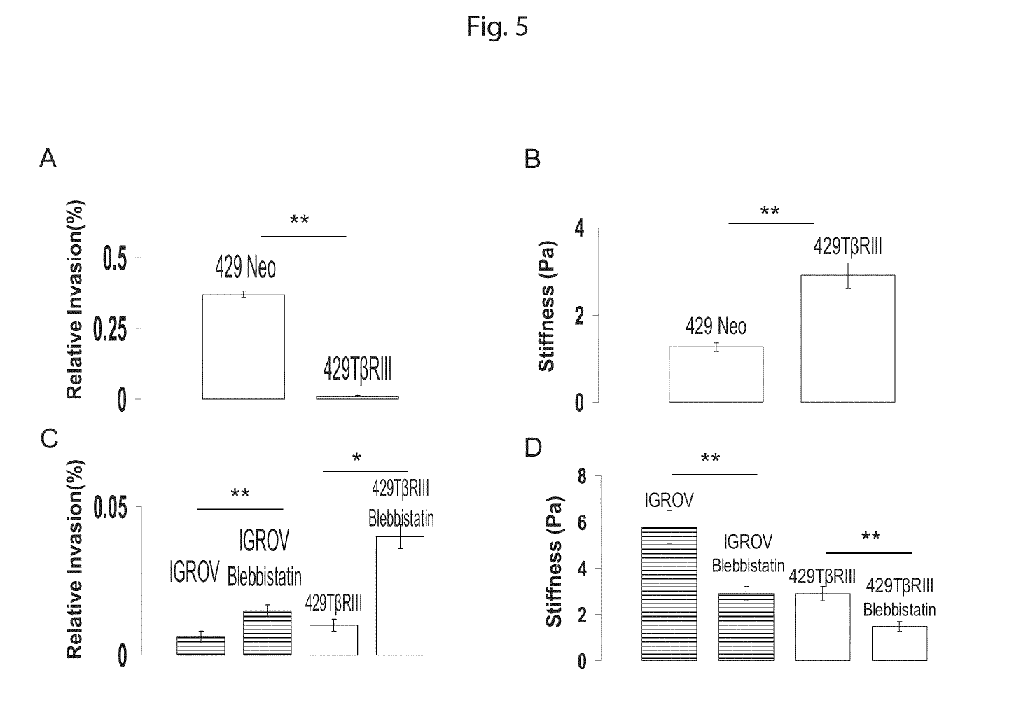

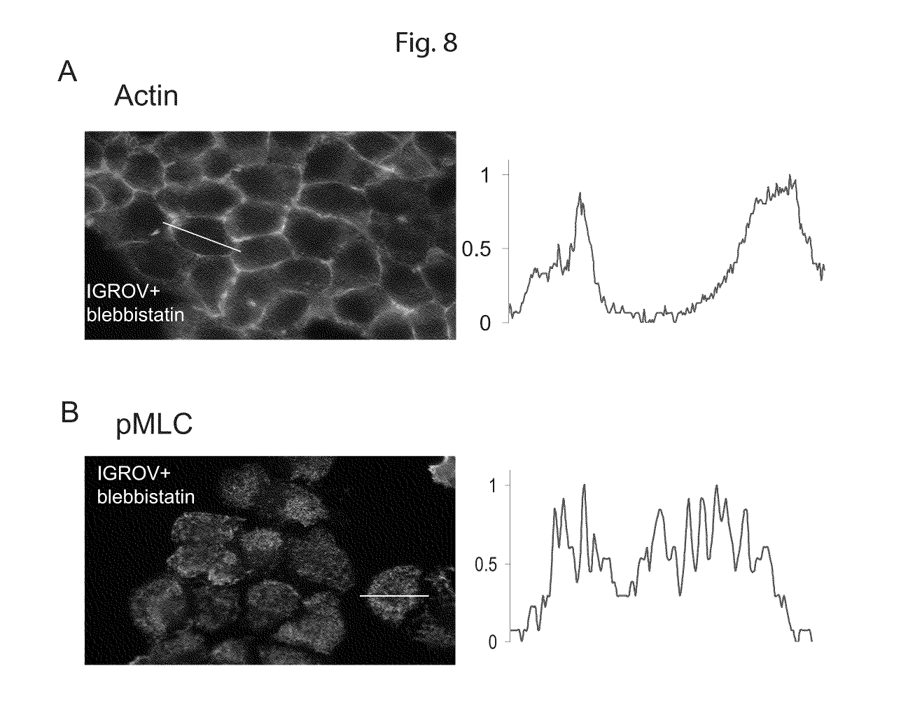

To investigate the role of stiffness as impacted by the cytoskeleton on migration and invasion, we determined the effect of altering acto-myosin contractility on these properties. Since cells with differential invasiveness (IGROV vs. HEY) had distinct pMLC localization and cytoskeletal architecture (FIG. 4), we used blebbistatin, a Myosin II inhibitor on the stiffest cell line (IGROV) and examined the effect on cell stiffness, migration and invasion. Blebbistatin, at a concentration that disrupted cortical pMLC localization but did not affect viability, increased cell invasion by 2.5 fold, cell migration by 4 fold and decreased cell stiffness by 2 fold (FIGS. 5C and 5D). We also observed concomitant alterations in the actin cytoskeleton of IGROV cells (FIG. 8), supporting a relationship between actomyosin contractility, cell stiffness and invasion of cancer cells. Another factor implicated in regulating migration and invasion either via the cytoskeleton or via secretion of proteases like MMPs is transforming growth factor-β (TGF-β)¹⁸. Early in carcinogenesis tumours become resistant to the homeostatic effects of TGF-β. One of the proposed mechanisms is loss of expression of the type III TGF β receptor (TβRIII), which has been demonstrated in a number of human cancers, including cancers of the breast, lung ovary, pancreas and prostate (reviewed in ¹⁸). TβRIII has been demonstrated to regulate cancer cell motility via alterations in the actin cytoskeleton⁹. To further investigate the contribution of the cytoskeleton and stiffness on cancer cell invasion, we examined the effect of TβRIII expression on cancer cell stiffness using stable cell lines Ovca429Neo, (no TβRIII expression), and Ovca429TβRIII, (TβRIII expression restored)⁹. We found that Ovca429TβRIII cells were two-fold stiffer than Ovca429Neo cells (K_{ovca429-TβRIII}=1.29 Pa; FIG. 5B). Further, this increase in stiffness corresponded to a decrease in invasiveness, similar to the correlation observed in the cancer cell lines (FIG. 5A). In addition, treating Ovca429TβRIII cells with blebbistatin increased their invasiveness by four fold and decreased the stiffness by two fold (K_{ovca429-TβRIII-blebbistatin}=1.48 Pa, I_{ovca429-TβRIII-blebbistatin}0.04%) similar to effects seen with blebbistatin treatment of the stiffest ovarian cancer cell line, IGROV (FIGS. 5C and 5D). Hence, cytoskeletal stiffness and effects on myosin II function may mediate suppression of migration and invasion by TβRIII.

Our results are the first evidence that metastatic potential measured through cancer cell invasion shows an inverse power-law relationship with cell stiffness. The particular exponent we derive may depend on the methodology employed for mechanical property determination. As cancer cells get progressively more invasive, they display softer mechanical characteristics that result in cell deformation and shape changes suitable for a metastatic population. We also find that cell lines having similar cytomorphology and cells from patients with similar stage disease can have widely different invasive potential that correlates with differences in stiffness. Currently, cell based diagnoses in cancer rely on histology examination of the removed tissue sample through antibody labeling of specific markers. This complex process is not always reliable and lacks quantified assessment of the disease state. Hence, sensitive biophysical measurements such as those demonstrated here, can be performed in short periods of time, on samples obtained from either ascites or circulating cells, providing potentially unique information about the patient's cancer including metastatic potential. Application of more sophisticated models to quantify scale free cell mechanics will provide further insight into this relationship^{19,20}. These insights into biomechanical changes during cancer progression have the potential to lead to novel therapy for treatments. Our observation that the relationship between invasiveness and stiffness is maintained across a series of cancer cell lines, in patient tumor specimens, and under cell biochemical modifications that increase and decrease cell stiffness, suggests that magnetic bead assays for stiffness may be a clinically applicable predictor of invasive potential, and that treatments that affect cellular stiffness, independent of mechanism, may be useful anti-metastatic approaches.

We claim:

1. A method of predicting the invasiveness or metastatic potential of a cancer cell comprising: (a) determining the creep compliance (deformability) or the spring constant (stiffness) of the cancer cell and (b) predicting the invasiveness or metastatic potential of the cancer cell based on the determination of step (a), wherein the creep compliance of the cell is proportional to the invasiveness and the metastatic potential of the cell and the spring constant is inversely proportional to the invasiveness and the metastatic potential of the cell.

2. The method of claim 1, wherein the prediction of step (b) is used to provide a prognosis to a subject from whom the cancer cell was obtained.

3. The method of claim 1, wherein the creep compliance or the spring constant is determined using magnetic tweezers, atomic force microscopy, micropipette aspiration, microfluidic optical stretcher, laser or optical tweezers, shear flow, substrate stretcher or a microplate stretcher.

4. The method of claim 1, wherein the cancer cells were obtained from a subject from biopsy, ascites, tumor, urine, sputum, pleural fluid or circulating cells.

5. The method of claim 1, wherein the cancer cells are obtained from a solid tumor.

6. The method of claim 1, wherein the cancer cells are ovarian, breast, uterine, lung, colon, pancreatic, prostate, stomach, thyroid, skin, melanoma, liver, esophagus, head and neck, bladder, sarcoma, cervical or kidney cancer cells.

7. The method of claim 1, wherein a creep compliance (Jₘₐₓ) greater than 1 correlates with increased invasiveness and metastatic potential.

8. The method of claim 7, wherein the creep compliance is greater than 2.

9. The method of claim 1, wherein a spring constant (k) of less than 2 correlates with increased invasiveness and metastatic potential.

10. The method of claim 9, wherein the spring constant is less than 1.

11. The method of claim 1, further comprising determining a treatment regimen for a subject.

12. A method of screening for an agent capable of reducing the invasiveness of a cancer cell comprising: contacting the cancer cell with the agent and determining the creep compliance or spring constant of the cell after contact with the agent, wherein an agent capable of decreasing the creep compliance or increasing the spring constant of the cell as compared to a second cancer cell not contacted with the agent is an agent capable of reducing the invasiveness of the cancer cell.

13. The method of claim 12, wherein the creep compliance or the spring constant is determined using magnetic tweezers, atomic force microscopy, micropipette aspiration, microfluidic optical stretcher, laser or optical tweezers, shear flow, substrate stretcher or a microplate stretcher.

14. The method of claim 12, wherein the cancer cells are primary cells or cell lines known to be invasive.

15. The method of claim 12, wherein the cancer cells are ovarian, breast, uterine, lung, colon, pancreatic, prostate, stomach, thyroid, skin, melanoma, liver, esophagus, head and neck, bladder, sarcoma, cervical or kidney cancer cells.

16. A method of screening for cancer cells or diagnosing cancer in a subject comprising measuring the creep compliance (deformability) or the spring constant (stiffness) of a cell from the subject to predict whether the cell is cancerous, wherein high creep compliance of the cell is predictive of the cell being cancerous and low spring constant is predictive of the cell being cancerous.

17. The method of claim 16, wherein the subject has been treated for cancer prior to the method.

18. The method of claim 16, wherein the subject is at risk of developing cancer or is suspected of having cancer.
